(19) United States
(12) Patent Application Publication
(10) Pub. No.: US 2025/0213313 A1
(43) Pub. Date: Jul. 3, 2025

(54) ROBOTIC CONTROL FOR MULTIPLE STEERABLE CATHETERS

(71) Applicant: Siemens Medical Solutions USA, Inc., Malvern, PA (US)

(72) Inventors: Young-Ho Kim, West Windsor, NJ (US); Jarrod Collins, Wilsonville, OR (US); Tommaso Mansi, Plainsboro, NJ (US)

(21) Appl. No.: 18/848,487

(22) PCT Filed: Apr. 13, 2023
(86) PCT No.: PCT/US2023/018494

§ 371 Date: Sep. 18, 2024

Related U.S. Application Data
(60) Provisional application No. 63/363,331, filed on Apr. 21, 2022.

(51) Int. Cl. A61B 34/30

(52) U.S. Cl. CPC A61B 34/30; A61B 2034/301

(57) ABSTRACT

For robotically operating multiple catheters, a common interface is used for control of two or more different catheter robotics systems. Information is used from one catheter robotic system for control of the other robotic catheter system. The common interface provides for collaborative control. Since different robotic systems may be used for different catheters, the common interface allows for the same user input and control to be translated for robotic control using any of various catheters.

### RELATED APPLICATION

The present patent document claims the benefit of the filing date under 35 U.S.C. § 119 (e) of Provisional U.S. Patent Application Ser. No. 63/363,331, filed Apr. 21, 2022, which is hereby incorporated by reference.

### BACKGROUND

The present embodiments relate to robotic control of medical catheters. One example medical catheter is an intracardiac echocardiography (ICE) catheter, which is used for cardiac interventional and diagnostic procedures. ICE is able to provide close feedback of anatomical structures and tools during a surgical procedure. Other catheters include ablation, optical imaging, guidance, stent placement, needle, or therapeutic.

Advances in polymers, steerable catheters, and medical robotics enable complex minimally invasive surgeries (MIS). The benefits of MIS are well-known: improved patient safety, reduced hospitalization, and reduced cost overall. However, MIS comes with an increase in workflow complexity and requires specific operator skills, leading to long training times. Robotic systems aim to simplify procedures through robotic control and make steerable endoscope and catheter handling more precise. Yet, these robotic systems target only very specific devices or catheters. Procedure steps that require other devices are still done manually by the operator. For example, a robotic system allows control of an ablation or mapping catheter especially designed for that system with limited ICE catheter manipulation. Other devices still need to be manipulated manually.

### SUMMARY

By way of introduction, the preferred embodiments described below include methods, systems, and robots for robotically operating multiple catheters. A common interface is used for control of two or more different catheter robotics systems. Information is used from one catheter robotic system for control of the other robotic catheter system. The common interface provides for collaborative control. Since different robotic systems may be used for different catheters, the common interface allows for the same user input and control to be translated for robotic control using any of various catheters.

In a first aspect, a multi-control system is provided. A first robotic catheter system is provided for a first steerable catheter. A second robotic catheter system is provided for a second steerable catheter. An interface processor is configured to generate a control interface for control of both the first and second robotic catheter systems.

In one implementation, the first steerable catheter is an intracardiac echocardiography catheter. The first robotic catheter system is configured to robotically control the intracardiac echocardiography catheter.

In another implementation, the first and second robotic catheter systems have first and second bases having motors for steering the first and second steerable catheters. The first base is separate from the second base and includes first and second adaptors connected with the first and second bases, respectively. The first and second adaptors are configured to hold the first and second steerable catheters respectively. Alternatively, the first and second robotic catheter systems have a shared base with motors for steering the first and second steerable catheters.

In yet another implementation, the interface processor is configured to communicate from the control interface to robot interfaces for the first and second robotic catheter systems. The control interface is configured to receive input from the user for the control of both the first and second robotic catheter systems, and the robot interfaces are configured to convert the input to instructions for operating motors of the first and second robotic catheter systems.

As an implementation, the interface processor is configured as a common interface for the control of both the first and second robotic catheter systems and as first and second interfaces for control of the first and second robotic catheter systems, respectively, based on the control from the common interface.

According to one implementation, the interface processor is configured to generate the control interface for user input of the control for both of the first and second robotic catheter systems in a same way.

In another implementation, the interface processor is configured to use an image from an intracardiac echocardiography catheter for image-based control of the second steerable catheter by the second robotic catheter system. For example, the image-based control includes (a) updating kinematics of the second robotic catheter system based on detection of the second steerable catheter in the image, (b) updating a Jacobian matrix for the second robotic catheter system based on a mismatch between a predicted position of the second steerable catheter and an observed position of the second steerable catheter in the image, and/or (c) updating a hysteresis model based on the image.

In one implementation, the interface processor is configured for calibration of the first and second robotic catheter systems and co-registration of the first robotic catheter system with the second robotic catheter system. For example, the interface processor is configured to co-register by kinematics update of the first robotic catheter system relative to the second robotic catheter system with respect to a known tip position of the second steerable catheter.

According to another implementation, the interface processor is configured to avoid collusion between the first and second steerable catheters.

According to yet another implementation, the interface processor is in a different room than the first and second robotic catheter systems.

In an implementation, the interface processor is configured to use an image from the first steerable catheter for automatic control by the second robotic catheter system of the second steerable catheter.

As one implementation, the interface processor is configured to automatically control the first robotic catheter system to steer the first steerable catheter system to image the second steerable catheter as the second steerable catheter is steered by the second robotic catheter system based on the control from the interface processor.

In a second aspect, a method is provided for robotically operating first and second catheters. The first and second catheters are attached to one or more bases on a patient bed or rail. A common interface is generated for robotic control of both the first and second catheters. The first and second catheters are robotically manipulated together using the common interface. The manipulation uses information from the first catheter for control of the second catheter.

According to one implementation, the common interface is generated using a same user input for the robotic manipulation of both the first and second catheters. The same user input is in a coordinate system of the patient.

As another implementation, robotic manipulation includes sending first control for the first catheter from the common interface to a first catheter robotic control and sending second control for the second catheter from the common interface to a second catheter robotic control.

In another implementation, robotic manipulation includes using imaging from the first catheter for control of the second catheter.

In a third aspect, a catheter control system is provided. A memory is configured to store instructions. A processor is configured by the instructions to: manipulate a first catheter robot for a first steerable catheter; and manipulate a second catheter robot for a second steerable catheter. The manipulation of the second catheter robot is in collaboration with the manipulation of the first catheter robot.

In example implementations, the collaboration includes calibration, co-registration, kinematics and/or Jacobian update, common patient coordinate system, collision avoidance, ablation targeting from imaging, imaging following, contact force estimation, treatment coverage, and/or image reconstruction.

The present invention is defined by the following claims, and nothing in this section should be taken as a limitation on those claims. Features of one aspect or type of claim (e.g., method or system) may be used in other aspects or types of claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a multi-control system for multiple steerable catheters;

FIG. 2 illustrates one embodiment of an interface configuration for control of multiple catheters; and

FIG. 3 is a flow chart diagram of one embodiment of a method for robotically operating multiple catheters.

### DETAILED DESCRIPTION OF THE DRAWINGS AND PRESENTLY PREFERRED EMBODIMENTS

Autonomous robotic control of multiple steerable catheters leverages information from or for the multiple catheters. A robotic platform controls and manipulates steerable catheters and leverages a multi-robot setup for collaborative robotic manipulation and automation. The limited application range of medical robots is expanded to support more extensive parts of the procedure and robotically control a variety of steerable catheters. Robotic manipulation of multiple steerable catheters at the same time is provided in a compact form factor. For an ICE example, one robot manipulates an ICE catheter, and another robot manipulates a needle for transseptal puncture or an ablation catheter. The two robots would work collaboratively to enable advanced features like continuous monitoring of the needle or ablation catheter.

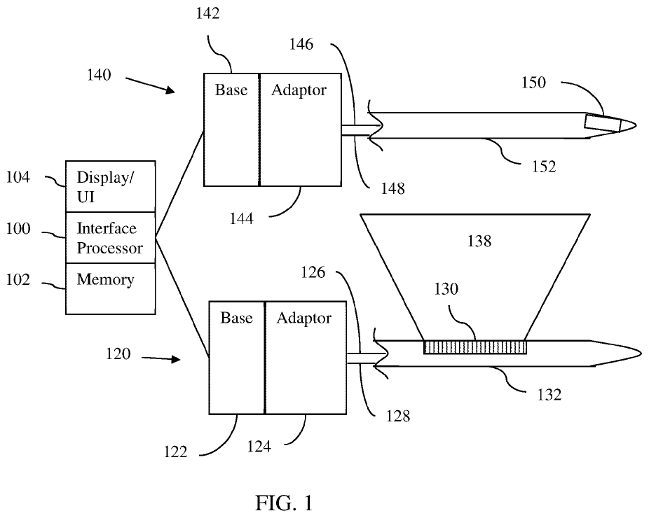

FIG. 1 is a block diagram of one approach for a catheter control system. The catheter control system controls multiple devices, so is a multi-control system. The multi-control system controls two or more devices. A common interface and/or user interface is provided for interacting with the user, operator, and/or processes and communication with the different devices.

In the approach of FIG. 1, two robotic catheter systems 120, 140 are controlled by the common interface formed by the interface processor 100, memory 102, and display and user interface 104. Additional, different, or fewer components may be provided, such as providing a third, fourth, or more controlled devices (e.g., robotic catheter systems and/or other robotic systems).

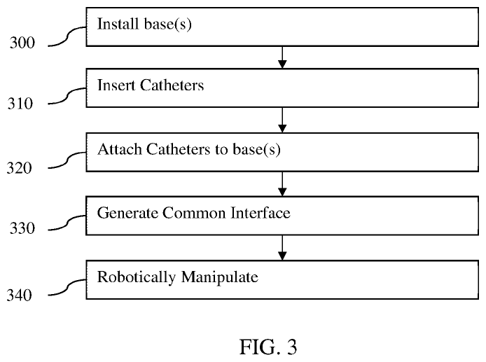

The catheter control system uses the common interface to interact with the multiple robotic catheter systems 120, 140 for collaborative operation. Information from one robotic catheter system 120 may be used for controlling operation of another robotic catheter system 140 and/or information from both may be used together for catheterization treatment and/or imaging of the patient. Information may be exchanged and/or common information used to control both robotic catheter systems 120, 140. In one approach, the catheter control system implements the method of FIG. 3 and/or another method.

The robotic catheter systems 120, 140 are the same or different design. For example, the robotic catheter systems 120, 140 are configured for robotically controlling the same or different types of catheters 132, 152. In an example used herein, the robotic catheter system 120 is configured to control an ICE catheter 132, and the robotic catheter system 140 is configured to control an ablation catheter 152. Other types of steerable catheters may be used. Other catheter-like devices (e.g., fiber optic endoscope or needle) may be used. These different catheters 132, 152 may have the same or different arrangements of control knobs, steering, degrees of freedom, and/or operation, so the robotic catheter systems 120, 140 are configured to operate or control arrangements of the catheters 132, 152 being used. Different catheter systems 120, 140 may be configured to operate with different types of catheters and not other types.

Any type or design of robotic catheter systems 120, 140 may be used. In the example herein, each robotic catheter system 120, 140 includes a base 122, 142, and an adaptor 124, 144. The base 122, 142 includes the motors for steering and/or operating the respective catheter 132, 152, and the adaptor 124, 144 is configured to mate with, hold, and/or manipulate the catheter 132, 152. For example, the adaptor 124, 144 allows the robotic catheter system 120, 140 to operate a steerable catheter 132, 152 designed for manual control by a physician. U.S. Pat. No. 11,590,319, the disclosure of which is incorporated herein by reference, discloses various embodiments of such a robotic catheter system. Other designs may be used, such as robotic catheter systems designed to robotically control a catheter designed specifically for or to mate with and be controlled by the robot (e.g., no adaptor 144).

The base 122, 142 includes motors to manipulate the steerable catheter 132, 152 in any number of degrees of freedom, such as 4 or more degrees of freedom (e.g., global translation, global rotation, and bending or steering the catheter 132, 152 in two or more directions). Gearing, motors, grippers (clamps), connectors, and/or other robotic components are included the base 122, 142 for generating and transmitting force to operate the catheter 132, 152. The robotic catheter systems 120, 140 apply push and/or pull forces to steering wires 126, 128, 146, 148 (e.g., three or four steering wires per catheter 132, 152) to operate the catheters 132, 152. The tips 136, 156 are steered for catheter operation. Electrical signals, translation, and/or rotation force may be generated in the base 122, 142. Global translation and/or rotation forces may similarly be applied, such as through the handles of the catheters 132, 152.

FIG. 1 shows a separate base 122, 142 for each robotic catheter system 120, 140. Each base 122, 142 is used to control or operate the respective catheter 132, 152. One base 122 can control one catheter at a time, with fast release with the adaptor 124. To manipulate several catheters 132, 152 at the same time, multiple and/or separate bases 122, 142 are used. The bases 122, 142 attach to the bed and/or rails of the patient operating table. The bases 122, 142 may instead be free standing, attach to rails on the floor, walls, or ceiling and/or may connect with robotic arms.

In other approaches, a shared base is used. The housing and/or frame for the shared base supports motors for operating the different adaptors 124, 144 and/or steerable catheters 132, 152. For example, the base 142 of one robotic catheter system 140 is expanded or altered to host (connect with and/or include) the base 122 of the other robotic catheter system 120.

The adaptor 124, 144 connects or plugs to the base 122, 142 and manipulates the catheter 132, 152. In one embodiment, the adaptors 124, 144 are clamshells or other arrangement of housing, gearing, connectors, and/or other robotic components to transfer force from the base 122, 142 to the catheter 132, 152 to steer and/or otherwise operate the catheter 132, 152. In one approach, the adaptor 124, 144 fits around the handle and/or body of the catheter 132, 152 to allow for manipulating the actuators (e.g., knobs) of the catheter 132, 152, for instance AP/LR knobs of an ICE catheter and/or the handle of the catheter 132, 152 for global translation and/or rotation (e.g., push-pull knob of an ablation catheter, etc.). Other adaptors 124, 144 that support other degrees of freedom (e.g., 2, 3, 4, or more) may be used, such as for control of needle or ablation catheters.

Where one of the steerable catheters 132 is an ICE catheter, the robotic catheter system 120 may be configured to control the ICE catheter, including imaging. The array 130 of elements is controlled through beamforming to generate ultrasound images. The imaging control may instead be part of the common interface or control. The field of view 138, type of imaging, and/or other imaging control may be provided. Where one of the steerable catheters 152 is an ablation or other treatment catheter, the robotic catheter system 140 may be configured to control the ablation or treatment catheter. For example, the contact sensing and/or electrode or other ablation applicator 150 is controlled to ablate or treat. The ablation control may be performed by the robotic catheter system and/or the common interface.

Where different types of catheters 132, 152 may attach to a same robotic catheter system 120, 140 (e.g., to the adaptor 124, 144), the robotic catheter system 120, 140 may identify or recognize the type of attached catheter 132, 152. The user interface 104 may allow for user selection to indicate the attached catheter 132, 152. Alternatively, a QR code on the box of the catheter 132, 152 and/or an RFID tag in the catheter 132, 152 is read to identify the type of catheter 132, 152. The identity of the catheter 132, 152 is communicated to the common interface, which uses the identify to register the catheter 132, 152. The control software for that catheter 132, 152 and corresponding robotic catheter system 120, 140 is loaded from the memory 102 or accessed from a database so that the common interface includes the software for operation of the robotic control system 120, 140 with the identified catheter 132, 152. Alternatively, a processor and control software are part of the robotic catheter system 120, 140, and the common interface loads the format and communications for interacting to control the robotic control system 120, 140 for the registered catheter 132, 152.

The common interface or control includes the interface processor 100, the memory 102, and the display/user interface 104. In one implementation, the interface processor 100 and memory 102, with or without the display/user interface 104 are part of a computer or server. Additional, different, or fewer components may be provided. For example, the memory 102 is not provided.

The common interface may be in a same room as the robotic catheter systems 120, 140. Alternatively, part (e.g., processor 100 and/or memory 102) or all the common interface is remote from the robotic catheter systems 120, 140 and patient, such as in a different room, different building, different facility, and/or different region (county, zip code, state, and/or country). Cloud-based computing by a remote server may be used. The user interface/display 104 may also be remote or be local to the patient.

The memory 102 is a non-transitory memory, such as a removable storage medium, a random access memory, a read only memory, a memory of a field programmable gate array, a cache, and/or another memory. The memory 102 is configured by a processor, such as the interface processor 100, to store control signals, information, control software, artificial intelligence, instructions executable by the interface processor 100, instructions executable by the robotic catheter systems 120, 140, interface software, and/or other information.

The display/user interface 104 is a display screen, such as a liquid crystal display or monitor, and a user input, such as a keyboard, knobs, sliders, touchpad, mouse, and/or trackpad. The display/user interface 104 may include icons, graphics or other imaging, such as from an operating system or application, for user interaction (selection, activation, and/or output).

The interface processor 100 is a general processor, application specific integrated circuit, integrated circuit, digital signal processor, field programmable gate array, artificial intelligence processor, tensor processor, and/or other controller for interfacing with the robotic catheter systems 120, 140, the user interface/display 104, and/or catheters 132, 152. The interface processor 100 is configured by design, hardware, and/or software to interface between the user and multiple robotic catheter systems, between the common interface and the user, and/or between the common interface and the robotic catheter systems 120, 140. Multiple processors may be used for sequential and/or parallel processing as the interface processor 100. The instructions from the memory 102, when executed by the processor 100, cause the processor 100 to operate the robotic catheter systems 120, 140 and/or the steerable catheters 132, 152 and to interface between various components and/or with the user or operator.

The interface processor 100 is located in a same room as the robotic catheter systems 120, 140. For example, a computer or workstation is connected with wires or wirelessly for interacting with the robotic catheter systems 120, 140. In one implementation, the interface processor 100 is a processor of one of the robotic catheter systems 120, 140. Alternatively, the interface processor 100 is in a different room than the robotic catheter systems 120, 140, such as in a room of the same building, different building, different facility, or different region. Computer network communications are used between the interface processor 100 and the robotic catheter systems 120, 140. The control through the interface processor 100 for both the robotic catheter systems 120, 140 allows the interface processor 100 to be fully remote from the procedure, patient, and/or robotic catheter systems 120, 140 as the interface processor 100 allows all catheters 132, 152 to be manipulated robotically. For cardiac electrophysiology for instance, two bases 122, 142 are used, one for the ICE catheter 132 to continuously monitor the procedure, the second one to manipulate a steerable sheet in which the needle is used and an ablation catheter 152. A supporting nurse may be in the lab to support catheter switching on the.

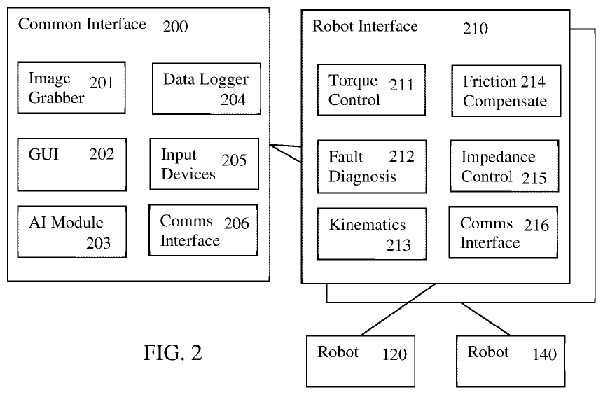

In one approach, different software interfaces, modules or layers are used for the common interface, such as a higher-level common interface and separate lower-level robot interfaces. FIG. 2 shows an example. A lower-level interface or robot interface 210 controls the robotic catheter system 120, 140 and communications between the common interface 200 and the robotic catheter system 120, 140. For example, the robot interface 210 is executed by the interface processor 100 and/or a processor of the robotic catheter system 120, 140. The robot interface 210 includes kinematics 213, torque control 211 of motors, fault diagnosis 212, impedance control 215, and/or communications interface 216. The robot interface 210 controls operation of the motors and/or robotic control system 120, 140, such as controlling for desired operation of the catheter 132, 152 and translating the control into instructions for the motors. Additional, different, or fewer instructions or operations for control of the robotic catheter system 120, 140 may be provided.

A different robot interface 210 is provided for each robotic catheter system 120, 140. Based on user input or signals (e.g., RFID) sent by the mechanical adaptor 124, 144, the low-level robot interface 210 is selected and loaded to control the robot and sensors. The low-level robot interface 210 is modular, with catheter-specific controllers that are enabled automatically upon registration.

The common interface 200 is a higher-level computing platform, software, and/or interface. The common interface 200 implements collaboration for joint operation of the catheters 132, 152 using information from one robotic catheter system 120, 140 for control of the other or both robotic catheter system 140, 120. The common interface 200 provides one graphic user interface 202 for a user to interact with, input control, and/or view information from the multiple robotic catheter systems 120, 140 and/or corresponding steerable catheters 132, 152. A common or uniform appearance and/or input options are provided for the different types of catheters 132, 152.

Various modules or instructions are provided for the common interface 200 for collaboration. For example, an image grabber 201 accesses one or more images generated by a catheter, such as ultrasound images. An artificial intelligence (AI) module 203 implements one or more machine-learned models, such as AI for automated control of one or both catheters 132, 152. The AI module 203 may additionally, or alternatively, implement one or more machine-learned models for classification, detection, and/or segmentation from one or more images captured by the image grabber 201. A data logger 204 accesses other information, such as from sensors, steering, calibration, registration, and/or feedback, from the GUI 202 and/or robot interfaces 210. The communications interface 206 formats control signals or information to communicate to the robot interface 210 and/or receives information from the robot interface 210. In one approach, the communications interface 206 formats communication using a computer network communications protocol, such as TCP/IP.

The common interface 200 controls the robotic catheter systems 120, 140 through the corresponding robot interfaces 210. The control is for one-by-one operation, such as sequential operation. The different robotic catheter systems 120, 140 may be simultaneously controlled. The control uses collaboration so that the catheters 132, 152 are operated jointly (in conjunction with each other) and/or controlled one based on information from another. The common interface 200, using the robot interfaces 210, manipulates one catheter robotic systems 120, 140 for one steerable catheter 132, 152 in collaboration with manipulation of the other catheter robotic system 140, 120 for the other steerable catheter 152, 132. Example collaboration includes calibration, co-registration, kinematics and/or Jacobian update, common patient coordinate system, collision avoidance, ablation targeting from imaging, catheter following with imaging, contact force estimation, treatment coverage, and/or image reconstruction. Additional, different, or fewer forms of collaboration may be provided.

In one implementation, the interface processor 100 is configured to generate a control interface for control of both the robotic catheter systems 120, 140. The display/user interface 104 provides inputs and outputs for operating the catheters 132, 152 via the robotic catheter systems 120, 140. The inputs and outputs are for user control of either or both of the catheters 132, 152. This control interface is the GUI 202 and input devices 205 of the common interface 200 for the control of both the robotic catheter systems 120, 140. The control instructions from the user, workflow process, and/or AI module 203 are provided to the robot interface 210 of the robotic catheter system 120, 140 being operated. The robotic catheter systems 120, 140 are operated by the respective robot interface 210 based on the control from the common interface 200. The control interface is configured to receive input from the user for the control of both the robotic catheter systems 120, 140, and the robot interfaces 210 are configured to convert the input to instructions for operating motors of the robotic catheter systems 120, 140 using the components of the robot interface 210.

The GUI 202 may present the controls for the different robotic catheter systems 120, 140 differently, such as modeling any differences in appearance of the user interfaces designed for the different robotic catheter systems 120, 140. Alternatively, the controls are generated on the GUI 202 for multiple of the different robotic catheter systems 120, 140 in a same way. The controls have a same look and feel, such as having the same options for available functions. Unavailable functions for a given robotic catheter system 120, 140 may be greyed out or not presented. For common controls, the same look, feel, and functionality are provided. Due to the common interface 200, all or multiple of the catheters 132, 152 can be manipulated in the x-y-z coordinate system of the patient and/or heart to improve hand-eye-coordination and ease of use. The operator manipulates, via the GUI 202, any steerable catheter 120, 140 in the same way, eliminating learning curves and risk of errors. This functionality could also enable new human-machine interactions, like virtually transporting the physician inside the body and manipulating, via the GUI 202, the catheters 132, 152 in a first-person perspective. The focus could also shift towards the target rather than the catheter 132, 152, for instance in a point-and-ablate fashion.

In one implementation using an ICE catheter (e.g., catheter 132), the interface processor 100 is configured to use an image from the ICE catheter for image-based control of the other steerable catheter 152 by the robotic catheter system 140. Vision-based algorithms further enhance robotic precision. The image-based control represents a collaboration between the two robotic catheter systems 120, 140 using the common interface 200. Example image-based control includes updating kinematics 213 of the robotic catheter system 140 based on detection of the steerable catheter 152 in the image from the ICE catheter. For example, the tip, fiducials, and/or other portion of the catheter 152 are identified by a detection AI of the AI module 203 from the ultrasound image. As another example, the shape of the ablation catheter 152 or needle is estimated from the ultrasound image using AI-based catheter tracing. The updated shape is used to automatically update the kinematics. The impedance control 215 may also be updated based on the shape for increased control precision. The relative spatial position between the array 130 and the detected catheter 152 is used to update or alter the kinematics. As another example, a Jacobian matrix for the robotic catheter system 140 is altered based on a mismatch between a predicted position of the steerable catheter 152 and an observed position of the steerable catheter 152 as detected in the image. By measuring the mismatch between predicted and observed position of the tip of the catheter 152, the underlying Jacobian matrix used for servoing and control can be automatically updated. In yet another example, a hysteresis model is updated based on the image. The underlying hysteresis model can be updated automatically periodically, or in real-time, using ICE imaging.

In another implementation for collaboration between the different robotic catheter systems 120, 140 using the common interface 200, the interface processor 100 is configured for calibration of the robotic catheter systems 120, 140 and/or co-registration of one robotic catheter system 120 with the other robotic catheter system 140. The calibration establishes a spatial location of the catheter 132, 152 in a coordinate system, such as the coordinate system oriented to the patient. The co-registration aligns the robotic catheter systems 120, 140 and/or corresponding catheters 132, 152 with each other. The co-registration may be by update of the kinematics 213 of one robotic catheter system 120 relative to the other robotic catheter system 140 with respect to a known tip position of the steerable catheter 152. The catheters may be automatically calibrated jointly, before or at given point in time during the procedure. For instance, the kinematics model of the catheter 152 is automatically updated using the images generated by the ICE catheter 132. Reversely, if the tip of the catheter 152 is fixed or in a known location, the kinematics 213 of the ICE catheter 132 may be refined automatically with respect to the fixed catheter tip.

As another example collaboration, the interface processor 100 is configured to avoid collision between the steerable catheters 132, 152. The robotic catheter systems 120, 140 are operated or controlled to avoid interference with each other. For example, having two or more robotically controlled catheters 132, 152 operated through the common interface 200 enables increased safety. Two or more catheters 132, 152 are controlled using the unified robotic controller for collision avoidance and multi-catheter path planning. The paths taken by each catheter 132, 152 may be used to build safety shells within which each robotic catheter system 120, 140 navigates. Monitoring to confirm avoidance or violation (entering the shell by the other catheter 132, 152) may be provided, in part, by imaging from the ICE catheter or pre-procedural imaging. In the case of cardiac electrophysiology, contact sensing information of a mapping device may be used as additional sensor information to create the safety shell. Contact being sensed is used to verify tip location relative to the patient. The interface processor 100 controls and/or limits control of the catheters 132, 152 to avoid one catheter 132, 152 entering the safety margin or shell of the other catheter 152, 132.

Other collaboration includes automated steering of the catheter(s) 132, 152. The interface processor 100 is configured to use an image from the ICE steerable catheter 132 for automatic control by the robotic catheter system 140 of the ablation steerable catheter 152. From the real-time ICE images, the processor 100, using the AI module 203 or other image detection, automatically finds the ablation target (e.g., target originally identified from preoperative CT or from anatomical landmark derived from AI-based auto-contouring). The ablation catheter 152 is driven by the interface processor 100, via the robotic catheter system 140, to place the electrode 140 or ablation device at the target. As both catheters are controlled by the same system, they are both co-registered and navigation is simplified.

For another example of collaboration, the ICE catheter 132 is automatically controlled to follow the ablation catheter 152 or needle. The interface processor 100 is configured to automatically control the robotic catheter system 120 to steer the ICE steerable catheter 132 to image the ablation steerable catheter 152 as the ablation steerable catheter 152 is steered by robotic catheter system 140. The interface processor 100 controls the robotic catheter systems 120, 140. The robotic catheter system 120 for the ICE catheter 132 is controlled to follow automatically, such as using an AI from the AI module 203 or another tracking process. The ablation catheter 152 is controlled by the interface processor 100 based on user input at the GUI 202 and/or based on automation. Both catheters 132, 152 are robotically controlled through the common interface 200. The ICE catheter 132 may automatically steer towards a specific view to optimize the visualization of an ablation from an optimal angle as the ablation catheter 152 is moved. This feature can be applied during a single ablation or during an “ablation line” as tissue is burned continuously. By recording the positions of ablation and ICE catheters 132, 152, the operator and/or interface processor 100 may quickly verify ablation coverage post ablation. The procedure may be replayed automatically for verification.

The servoing algorithm for movement to the ablation target and/or for ICE imaging following the ablation catheter 152 may rely on the ICE images, electromagnetic tracking at the tips and/or other position sensing of the catheters 132, 152, and/or robotic control information (e.g., motor information, kinematics 213, etc.). High accuracy is provided in targeting (ablation and transseptal puncture) and/or following due to the collaboration and/or common interface 200.

The interface processor 100 may be configured for vision-based contact force estimation. This contact force estimation may be an additional source of information for an electrophysiologist to assess proper contact during ablation. By ensuring the ICE catheter 132 tracks the ablation catheter, the shape of the ablation catheter 152 shown in the ultrasound image from the ICE catheter 132 may indicate the force applied by the catheter 152 to tissue. An AI from the AI module 203 or other detection process may estimate the force from the image.

In another example, joint control is provided for an ICE catheter 132 and fiber optics for opto-acoustic imaging. The robotic catheter system 140 controls the position of the fiber optics or endoscope to optically view the patient from within. When both are controlled by the interface processor 100, each device is co-registered, making opto-acoustic reconstruction possible. The opto-acoustic reconstruction forms a three-dimensional (3D) representation of the patient and/or catheters 132, 152 in both optical and ultrasound as aligned by the co-registration and/or forms aligned but separate 3D optical and 3D ultrasound representations.

Other collaborative processes may be implemented by the interface processor 100. To encourage device companies to rely on the system, an open-source application programming interface (API) and framework may be provided to develop the adaptors 124, 144 and implement the related low-level controller and kinematics models (e.g., the robot interface 210 for the interface processor 100). The API may also encourage sensor information exchange, which can benefit the robot in its autonomous tasks and also the device (catheter) should the device use specific information from imaging (e.g., energy calibration).

Any type of catheter may be used. Catheter-like devices, such as long flexible endoscope (fiber optics) or needles, may be used as one of the catheters.

FIG. 3 is a flow chart diagram of one embodiment of a method for robotically operating multiple catheters. Different robotic systems are operated together for control of different catheters. A common interface links the different robotic systems for collaborative control.

The method is implemented by the system and/or robotic systems of FIG. 1 or 2 or another system. Additional, different, or fewer acts may be provided. For example, acts 300, 310, and/or 320 are not provided. As another example, acts for the catheterization workflow are provided. In yet another example, acts for pre-operative planning and/or pre, during, and/or post procedure imaging are provided. As another example, acts for ablation, stent placement, or other catheter function are provided.

The acts are performed in the order shown (top to bottom or numerical) or a different order. For example, act 310 is performed before, after, or simultaneously with act 320. As another example, act 330 occurs prior to act 300, 310, and/or 320.

In act 300, a technician or operator installs the catheter robots. For example, the bases or base are clamped, clipped, or connected to the operating table (bed) or rail. The bases are positioned relative to the patient. Depending on the points of access of the catheters to the patient, the bases are positioned to align the catheters with the patient and point(s) of access.

In act 310, the physician inserts the catheters into the patient. The catheters are inserted through access point(s), such as through a trocar(s) in the patient's skin. The tip of each catheter is inserted into the patient and fed through the cardiovascular system to the region of interest (e.g., heart). In an alternative approach, act 320 is performed first and the robot is used to insert and/or feed the catheter in the patient using acts 330 and 340.

In act 320, the physician, operator, and/or technician attaches the catheters to the base(s) as installed on the bed or rail. Each catheter is attached to the respective base. Where a shared base is used, each catheter is attached to the respective part of the base.

The catheters are placed in the robotic systems. For example, each of the handles is placed in the adaptor for that catheter. The adaptor is closed around the handle and latched in place to hold the handle. Alternatively, the catheter is already attached to or part of the base.

This positioning occurs while the adaptor (holder) connects with the base. In one embodiment, the adaptor is separable from the base. The handle of the catheter is positioned in the adaptor while the adaptor is not attached to the base.

A sterile bag may be placed around the catheters, adaptors, and/or bases. In one approach, the base connects to the adaptor through a plastic interface of the sterile bag. The adaptor and the catheter are outside the sterile bag, and the base is in the sterile bag. Since all electronics are kept sterile by the bag, the electronics may be reused without cleaning or with a less intense cleaning. Since all parts not inside the bag are plastic or metal, these parts may be easily cleaned in-house and reused as many times as the catheter is reused.

In act 330, a processor generates a common interface for robotic control of both the catheters. A GUI is presented to the user, such as the physician. The same GUI may be used to control multiple different robots and corresponding catheters. A same user input is used for the robotic manipulation of multiple catheters. For example, the same controls are used for steering. The user selects the catheter to be steered on the GUI, and then steers that catheter. The user may select the other catheter and steer that catheter in the same way (i.e., same controls) even if the robot moves differently to implement the control. As another example, the user configures the robots to act according to a workflow, such as image tracking, registration, calibration, collision avoidance, and/or treatment region pathing. In yet another example, the user selects the workflow or catheterization process. The workflow or process for one or more catheters may be implemented automatically or semi-automatically.

The common interface may use a same coordinate system for each catheter. Since the catheters may be spatially registered or calibrated, the catheters may be controlled using the common coordinate system, such as the coordinate system of the patient.

In act 340, the processor robotically manipulates the catheters individually or together using the common interface. Based on workflow, process, user entered control, and/or other sources of desired operation of one or more catheters, the processor causes the robots to operate the catheters.

Controls are generated by the common interface. These controls are provided to lower level robot interfaces, which then control the robots to operate the catheters. For sequential operation, the common interface is used to switch the catheter being controlled at a given time. As the procedure occurs, the control may switch between the catheters multiple times. Alternatively, simultaneous control is used, such as the user inputting control (e.g., desired steering, movement, and/or other operation) for one catheter and the processor controlling the other catheter based on AI or a computer automated operation.

The common interface sends control or signals for the different catheters to the respective robot. The processor routes control from the common interface to robot interfaces, which control the robots. Different formats may be used for the controls for the different robots. The common interface allows control of the different robots despite the differences in format.

The control may use collaboration. For example, imaging from one catheter is used in the control of the other catheter. Calibration, co-registration, ablation region tracking, force application sensing, collision avoidance, multi-catheter pathing, catheter tracking or imaging, optimization of viewing angle for operation of a catheter (e.g., ablation), verification of ablation by imaging, and/or other collaboration between the catheters or robots for the catheters may be used.

The manipulation may include steering (e.g., bending in one or more planes), global rotation (e.g., rotation of the handle and corresponding catheter), global translation (e.g., pulling or pushing the catheter further out or further into the patient), imaging, ablation, puncture, inflation, stitching, clamping, cutting, pharmaceutical deposit, stent expansion, stent release, and/or other operation. The robots, under control from the common interface, operate the catheters.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A multi-control system comprising:
a first robotic catheter system for a first steerable catheter;
a second robotic catheter system for a second steerable catheter; and
an interface processor configured to generate a control interface for control of both the first and second robotic catheter systems.

2. The multi-control system of claim 1, wherein the first steerable catheter comprises an intracardiac echocardiography catheter, the first robotic catheter system configured to robotically control the intracardiac echocardiography catheter.

3. The multi-control system of claim 1, wherein the first and second robotic catheter systems comprise first and second bases having motors for steering the first and second steerable catheters, the first base separate from the second base, and comprise first and second adaptors connected with the first and second bases, respectively, the first and second adaptors configured to hold the first and second steerable catheters respectively.

4. The multi-control system of claim 1, wherein the first and second robotic catheter systems comprise a shared base with motors for steering the first and second steerable catheters.

5. The multi-control system of claim 1, wherein the interface processor is configured to communicate from the control interface to robot interfaces for the first and second robotic catheter systems, the control interface configured to receive input from the user for the control of both the first and second robotic catheter systems and the robot interfaces configured to convert the input to instructions for operating motors of the first and second robotic catheter systems.

6. The multi-control system of claim 1, wherein the interface processor is configured as a common interface for the control of both the first and second robotic catheter systems and as first and second interfaces for control of the first and second robotic catheter systems, respectively, based on the control from the common interface.

7. The multi-control system of claim 1, wherein the interface processor is configured to generate the control interface for user input of the control for both of the first and second robotic catheter systems in a same way.

8. The multi-control system of claim 2, wherein the interface processor is configured to use an image from the intracardiac echocardiography catheter for image-based control of the second steerable catheter by the second robotic catheter system.

9. The multi-control system of claim 8, wherein the image-based control comprises (a) updating kinematics of the second robotic catheter system based on detection of the second steerable catheter in the image, (b) updating a Jacobian matrix for the second robotic catheter system based on a mismatch between a predicted position of the second steerable catheter and an observed position of the second steerable catheter in the image, and/or (c) updating a hysteresis model based on the image.

10. The multi-control system of claim 1, wherein the interface processor is configured for calibration of the first and second robotic catheter systems and co-registration of the first robotic catheter system with the second robotic catheter system.

11. The multi-control system of claim 10, wherein the interface processor is configured to co-register by kinematics update of the first robotic catheter system relative to the second robotic catheter system with respect to a known tip position of the second steerable catheter.

12. The multi-control system of claim 1, wherein the interface processor is configured to avoid collusion between the first and second steerable catheters.

13. The multi-control system of claim 1, wherein the interface processor is in a different room than the first and second robotic catheter systems.

14. The multi-control system of claim 1, wherein the interface processor is configured to use an image from the first steerable catheter for automatic control by the second robotic catheter system of the second steerable catheter.

15. The multi-control system of claim 1, wherein the interface processor is configured to automatically control the first robotic catheter system to steer the first steerable catheter system to image the second steerable catheter as the second steerable catheter is steered by the second robotic catheter system based on the control from the interface processor.

16. A method for robotically operating first and second catheters, the method comprising:
attaching the first and second catheters to one or more bases on a patient bed or rail;
generating a common interface for robotic control of both the first and second catheters; and
robotically manipulating the first and second catheters together using the common interface, the manipulation using information from the first catheter for control of the second catheter.

17. The method of claim 16, wherein generating the common interface comprises using a same user input for the robotic manipulation of both the first and second catheters, the same user input being in a coordinate system of the patient.

18. The method of claim 16, wherein robotically manipulating comprises sending first control for the first catheter from the common interface to a first catheter robotic control and sending second control for the second catheter from the common interface to a second catheter robotic control.

19. The method of claim 16, wherein robotically manipulating comprises using imaging from the first catheter for control of the second catheter.

20. A catheter control system comprising:
a memory configured to store instructions; and
a processor configured by the instructions to:
manipulate a first catheter robot for a first steerable catheter; and
manipulate a second catheter robot for a second steerable catheter, the manipulation of the second catheter robot being in collaboration with the manipulation of the first catheter robot.

21. The catheter control system of claim 20, wherein the collaboration comprises calibration, co-registration, kinematics and/or Jacobian update, common patient coordinate system, collusion avoidance, ablation targeting from imaging, imaging following, contact force estimation, treatment coverage, and/or image reconstruction.
